(19) United States
(12) Patent Application Publication
(10) Pub. No.: US 2026/0162826 A1
(43) Pub. Date: Jun. 11, 2026

(54) LEARNING DEVICE, ANALYSIS DEVICE, ANALYSIS METHOD, AND NON-TRANSITORY STORAGE MEDIUM STORING PROGRAM THEREOF

(71) Applicant: FUKUDA DENSHI CO., LTD., Tokyo (JP)

(72) Inventors: Fumiaki ISERI, Tokyo (JP); Hiroki YAMAYA, Tokyo (JP)

(73) Assignee: FUKUDA DENSHI CO., LTD., Tokyo (JP)

(21) Appl. No.: 19/367,326

(22) Filed: Oct. 23, 2025

(30) Foreign Application Priority Data
Oct. 31, 2024 (JP) 2024-191671

(51) Int. Cl. G16H 50/20; A61B 5/00; A61B 5/347

(52) U.S. Cl. CPC G16H 50/20; A61B 5/347; A61B 5/7267

(57) ABSTRACT

A learning device includes a processor and a memory. The memory stores a program for causing the processor to execute acquiring first time-series data representing a biological waveform and a noise index representing a noise amount included in the first time-series data, generating a plurality of pieces of second time-series data different from each other by executing denoising or noise addition for the first time-series data, and causing a noise detection model to learn teaching data that includes data including the plurality of pieces of second time-series data as an input and includes the noise index as a correct answer.

### BACKGROUND

The present disclosure relates to a learning device, an analysis device, an analysis method, and a non-transitory storage medium storing a program thereof.

Arrhythmia such as atrial fibrillation can be found by analyzing electrocardiographic data measured by an electrocardiograph. The electrocardiographic data possibly includes noise attributed to body motion, electrical activity in muscle, or the like in a patient. The noise included in the electrocardiographic data adversely affects the finding of the arrhythmia. A technique for detecting the noise included in the electrocardiographic data is described in Japanese Translations of PCT for Patent No. 2017-525410 (hereinafter, Patent Document 1).

### SUMMARY

There is room for improvement in the noise detection described in Patent Document 1. An aspect of the present disclosure intends to provide a technique for accurately analyzing time-series data.

According to part of an embodiment, a learning device including a processor and a memory is provided. The memory stores a program for causing the processor to execute acquiring first time-series data representing a biological waveform and a noise index representing a noise amount included in the first time-series data, generating a plurality of pieces of second time-series data different from each other by executing denoising or noise addition for the first time-series data, and causing a noise detection model to learn teaching data that includes data including the plurality of pieces of second time-series data as an input and includes the noise index as a correct answer.

The time-series data can be accurately analyzed by the above-described configuration.

### BRIEF DESCRIPTION OF THE DRAWINGS

FIG. 1 is a block diagram for explaining a configuration of a computer according to part of the embodiment;

FIG. 2 is a flowchart for explaining a learning method for a noise detection model according to part of the embodiment;

FIG. 3 is a schematic diagram for explaining a generation method for input data to the noise detection model according to part of the embodiment;

FIG. 4 is a schematic diagram for explaining a configuration of the noise detection model according to part of the embodiment;

FIG. 5 is a flowchart for explaining a learning method for an arrhythmia detection model according to part of the embodiment;

FIG. 6 is a schematic diagram for explaining a generation method for input data to the arrhythmia detection model according to part of the embodiment;

FIG. 7 is a schematic diagram for explaining a configuration of the arrhythmia detection model according to part of the embodiment;

FIG. 8 is a flowchart for explaining an analysis method according to part of the embodiment; and

FIG. 9 is a schematic diagram for explaining a generation method for input data to each model according to part of the embodiment.

### DETAILED DESCRIPTION OF THE PREFERRED EMBODIMENT

An embodiment is described in detail below with reference to the accompanying drawings. The following embodiment does not limit the disclosure according to the scope of claims, and not all of combinations of features described in the embodiment are necessarily essential for the disclosure. Two or more features among a plurality of features described in the embodiment may be combined into any combination. Further, the same or similar configuration is given the same reference numeral, and overlapping description is omitted.

### Hardware Configuration of Computer

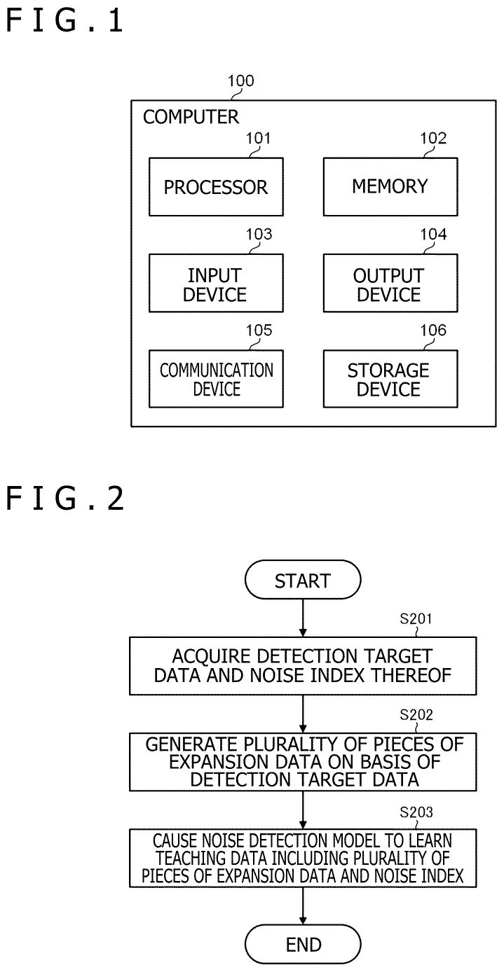

A configuration of a computer 100 according to part of the embodiment is described with reference to FIG. 1. As described in detail below, the computer 100 is used for executing machine learning. The computer 100 that executes the machine learning as above may be referred to as a learning device. Moreover, the computer 100 is used for analyzing a biological signal (for example, electrocardiographic waveform). The computer 100 that analyzes the biological signal as above may be referred to as an analysis device.

The computer 100 may include constituent elements depicted in FIG. 1. A processor 101 is a device that controls overall operation of the computer 100. The processor 101 may include, for example, a central processing unit (CPU) and a graphics processing unit (GPU). A memory 102 is a device that stores a program and temporary data used in the computer 100. The memory 102 includes, for example, a random access memory (RAM) and a read only memory (ROM). Operation of the computer 100 may be executed through, for example, execution of the program stored in the memory 102 by the processor 101. Instead of this, part or the whole of the operation of the computer 100 may be executed by a dedicated circuit such as an application specific integrated circuit (ASIC) or a field programmable gate array (FPGA).

An input device 103 is a device for acquiring an input from a user of the computer 100. The input device 103 includes, for example, a keyboard and a mouse. An output device 104 is a device for executing output for the user of the computer 100. The output device 104 includes, for example, a display and a speaker. A communication device 105 is a device for communication with another device by the computer 100. The other device may be a computer connected to a network (for example, the Internet or a local area network).

A storage device 106 is a device that stores data used for operation of the computer 100. The storage device 106 includes, for example, a storage medium such as a hard disk drive (HDD), a solid state drive (SSD), or a digital versatile disc (DVD).

### Learning of Noise Detection Model

With reference to FIG. 2, a description is given of a learning method for causing a model for detecting a noise amount included in time-series data of a detection target to learn teaching data. The learning of the model by a computer is referred to also as machine learning. In the following description, the time-series data of a detection target is represented as detection target data, and the model for detecting the noise amount included in the detection target data is represented as a noise detection model. The detection target data may be time-series data representing a waveform of a biological signal (that is, a biological waveform), and may be time-series data representing, for example, an electrocardiographic waveform, a pulse wave, a respiratory waveform, or the like. The noise detection model outputs an index representing the noise amount included in the detection target data. In the following description, the index representing the noise amount is represented as a noise index. The noise index may be represented with two levels (for example, “high” and “low”), or may be represented with three or more levels. Instead of this, the noise index may be represented by a numerical value proportional to the noise amount.

Each step in the method of FIG. 2 may be executed through execution of a program read out into the memory 102 by the processor 101. Instead of this, at least part of the steps in the method of FIG. 2 may be executed by a dedicated integrated circuit such as an ASIC or an FPGA. The method of FIG. 2 may be started in accordance with an instruction from the user of the computer 100.

In S201, the processor 101 acquires a dataset used in machine learning of the noise detection model. In the following description, the dataset used in machine learning is represented as a training dataset. The processor 101 may acquire the training dataset from an external server (for example, a database server) by using the communication device 105.

The training dataset for the noise detection model includes a plurality of pieces of training data. Each of the plurality of pieces of training data includes one piece of the detection target data (first time-series data) and the noise index representing the noise amount included in this detection target data. The detection target data is, for example, time-series data representing an electrocardiographic waveform measured by an electrocardiograph. The electrocardiograph that measures the electrocardiographic waveform may be either a Holter electrocardiograph or a polysomnography device, or may be an electrocardiograph of another type. The length of the detection target data is, for example, such a length as to include at least one time of pulsation, and may be, for example, approximately two seconds. The detection target data may be a part extracted from an electrocardiographic waveform measured for a long period (for example, a sleeping period or one day or longer). The noise index of the detection target data may be an index decided by a person, or may be an index decided through execution of specific processing by a machine.

In S202, the processor 101 generates a plurality of pieces of time-series data (second time-series data) different from each other on the basis of the detection target data concerning each piece of the training data of the training dataset acquired in S201. In the following description, the time-series data generated on the basis of the detection target data is represented as expansion data. Specifically, the processor 101 generates a plurality of pieces of expansion data different from each other by executing denoising or noise addition for the detection target data. The denoising refers to processing (for example, filtering) for reducing noise included in the detection target data. The noise addition refers to processing for increasing the noise included in the detection target data. The plurality of pieces of expansion data may include both the expansion data generated by executing the denoising for the detection target data and the expansion data generated by executing the noise addition for the detection target data, or may include only either one of them.

The plurality of pieces of expansion data may include both the expansion data generated by reducing a frequency component lower than a predetermined cutoff frequency (first cutoff frequency) from the detection target data and the expansion data generated by reducing a frequency component higher than a predetermined cutoff frequency (second cutoff frequency) from the detection target data, or may include only either one of them. These two cutoff frequencies may be the same, or may be different. Processing of reducing the frequency component lower than the predetermined cutoff frequency from the detection target data may be executed by applying a high-pass filter or a band-pass filter to the detection target data. Processing of reducing the frequency component higher than the predetermined cutoff frequency from the detection target data may be executed by applying a low-pass filter or a band-pass filter to the detection target data.

The plurality of pieces of expansion data may include a plurality of pieces of expansion data generated by reducing a frequency component lower than each of a plurality of cutoff frequencies different from each other from the detection target data as the expansion data generated by reducing the frequency component lower than the predetermined cutoff frequency from the detection target data. The plurality of pieces of expansion data may include a plurality of pieces of expansion data generated by reducing a frequency component higher than each of a plurality of cutoff frequencies different from each other from the detection target data as the expansion data generated by reducing the frequency component higher than the predetermined cutoff frequency from the detection target data.

The plurality of pieces of expansion data may include the expansion data generated by adding white noise to the detection target data as the expansion data generated by executing the noise addition for the detection target data. The plurality of pieces of expansion data may include a plurality of pieces of expansion data generated by adding white noise with amplitudes different from each other to the detection target data as the expansion data generated by executing the noise addition for the detection target data.

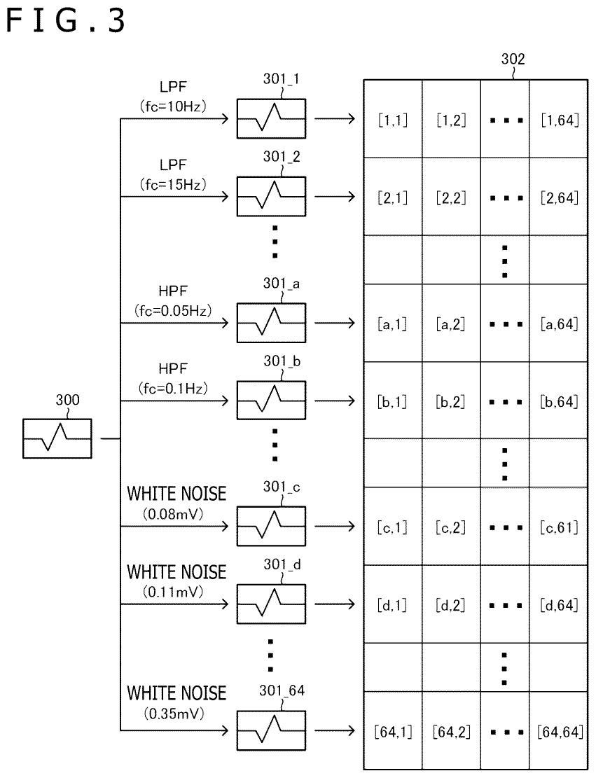

An example of detection target data 300 and pieces of expansion data 301_1 to 301_64 is described with reference to FIG. 3. In the example of FIGS. 3, 64 pieces of expansion data 301_1 to 301_64 are generated on the basis of one piece of detection target data 300. The number of generated pieces of the expansion data is not limited thereto. In the following description, the pieces of expansion data 301_1 to 301_64 are collectively represented as expansion data 301. Description concerning the expansion data 301 applies also to any of the pieces of expansion data 301_1 to 301_64.

The 64 pieces of expansion data 301 include time-series data resulting from reduction of a frequency component higher than a predetermined cutoff frequency (fc) from the detection target data 300 by a low-pass filter (LPF), time-series data resulting from reduction of a frequency component lower than the predetermined cutoff frequency (fc) from the detection target data 300 by a high-pass filter (HPF), and time-series data resulting from addition of white noise to the detection target data 300.

Specifically, the 64 pieces of expansion data 301 include a plurality of pieces of expansion data 301_1, 301_2, . . . generated by reducing a frequency component higher than each of a plurality of cutoff frequencies different from each other (for example, 10 Hz, 15 Hz, . . . ) from the detection target data 300 by using the LPF. Further, the 64 pieces of expansion data 301 include a plurality of pieces of expansion data 301_a, 301_b, . . . generated by reducing a frequency component lower than each of a plurality of cutoff frequencies different from each other (for example, 0.05 Hz, 0.1 Hz, . . . ) from the detection target data 300 by using the HPF. Moreover, the 64 pieces of expansion data 301 include a plurality of pieces of expansion data 301_c, 301_d, . . . , and 301_64 generated by adding white noise with amplitudes different from each other (for example, 0.08 mV, 0.11 mV, . . . and 0.35 mV) to the detection target data 300.

In S203, the processor 101 causes the noise detection model to learn teaching data that includes, as an input, data including the plurality of pieces of expansion data generated in S202 and includes the noise index as a correct answer concerning each piece of the training data of the training dataset acquired in S201. The data input to the noise detection model is referred to as input data. For example, the processor 101 calculates the difference between the noise index output from the noise detection model by inputting the input data to the noise detection model and the noise index of the correct answer concerning each piece of the training data of the training dataset, and updates a parameter of the noise detection model such that the sum of this difference across the plurality of pieces of the training data becomes small. Part of the plurality of pieces of the training data acquired in S201 may be used as validation data or test data of the noise detection model instead of being used as the teaching data.

An example of input data 302 to the noise detection model is described with reference to FIG. 3. The input data 302 is represented by a two-dimensional array. Each row of the input data 302 represents one of the plurality of pieces of expansion data 301 generated in S202. In each row of the input data 302, the expansion data 301 is represented by 64 signal values. Instead of this, one piece of the expansion data 301 may be represented by another number of signal values. For example, the 64 signal values are decided by sampling one piece of the expansion data 301 at a sampling cycle of 31.25 Hz.

In the example of FIG. 3, the number of columns and the number of rows of the input data 302 are equal to each other. Instead of this, the number of columns and the number of rows of the input data 302 may be different from each other. The input data 302 may include only one piece of the same expansion data, or may include a plurality of pieces of the same expansion data. In the example of FIG. 3, the input data 302 does not include the detection target data 300. Instead of this, the input data 302 may include the detection target data 300.

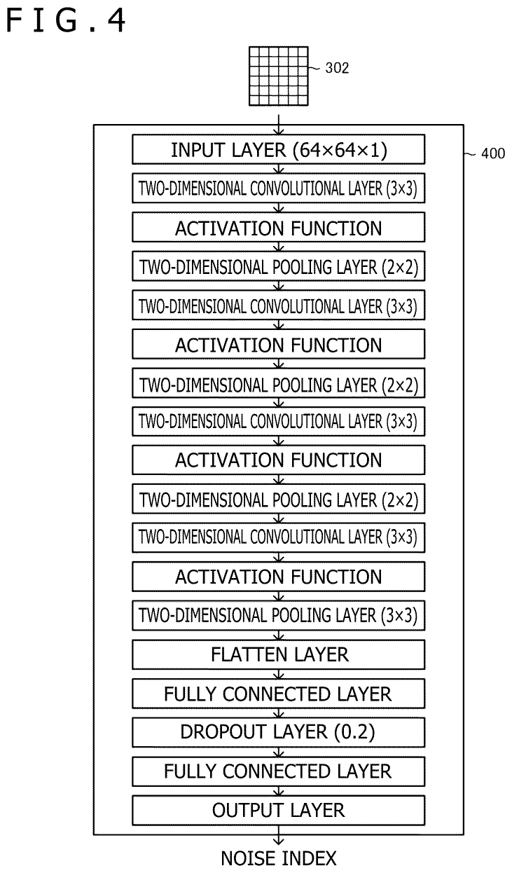

An example of a configuration of a noise detection model 400 is described with reference to FIG. 4. The noise detection model 400 includes a neural network (specifically, a convolutional neural network (CNN)). Instead of this, the noise detection model 400 may include another model such as logistic regression or a support vector machine. Numerical values in parentheses given to an input layer of the noise detection model 400 represent the size of the input data 302. Numerical values in parentheses given to two-dimensional convolutional layers and two-dimensional pooling layers of the noise detection model 400 represent the window size. A numerical value in parentheses given to a dropout layer of the noise detection model 400 represents the dropout probability. A specific configuration of each layer of the noise detection model 400 may be an existing configuration, and thus detailed description thereof is omitted.

In machine learning of the above-described noise detection model 400, the plurality of pieces of expansion data 301 different from each other in the noise amount are used as the one piece of input data 302. By training the noise detection model 400 by using such input data 302, the noise detection model 400 with high accuracy of detection of noise can be generated.

The plurality of pieces of detection target data included in the training dataset acquired in the above-described S201 may represent an electrocardiographic waveform of a single lead, or may represent electrocardiographic waveforms of a plurality of leads. For example, a Holter electrocardiograph can simultaneously measure an electrocardiographic waveform of the CM5 lead and an electrocardiographic waveform of the National Aeronautics and Space Administration (NASA) lead concerning the same patient. The learning method of FIG. 2 may be individually executed concerning each of the electrocardiographic waveform of the CM5 lead and the electrocardiographic waveform of the NASA lead. In this case, a noise detection model for outputting the noise index of the electrocardiographic waveform of the CM5 lead and a noise detection model for outputting the noise index of the electrocardiographic waveform of the NASA lead are separately generated. The plurality of leads may include other leads, for example, the CM2 lead, the CS2 lead, the CC5 lead, and the like, instead of the above-described CM5 lead and NASA lead or in addition to them.

Instead of this, a single noise detection model may be trained by using both the electrocardiographic waveform of the CM5 lead and the electrocardiographic waveform of the NASA lead. For example, the training dataset acquired in S201 may include training data including the detection target data (first time-series data) representing the electrocardiographic waveform of the CM5 lead and training data including the detection target data (third time-series data) representing the electrocardiographic waveform of the NASA lead. In the above-described S202, the processor 101 generates a plurality of pieces of expansion data (second time-series data) different from each other on the basis of the detection target data (first time-series data) of the electrocardiographic waveform of the CM5 lead, and generates a plurality of pieces of expansion data (fourth time-series data) different from each other on the basis of the detection target data (third time-series data) of the electrocardiographic waveform of the NASA lead. In the above-described S203, the processor 101 causes the single noise detection model to learn teaching data (first teaching data) that includes, as an input, data including a plurality of pieces of expansion data generated concerning the CM5 lead and includes the noise index (first noise index) of the electrocardiographic waveform of the CM5 lead as a correct answer and teaching data (second teaching data) that includes, as an input, data including a plurality of pieces of expansion data generated concerning the NASA lead and includes the noise index (second noise index) of the electrocardiographic waveform of the NASA lead as a correct answer. By causing the single noise detection model to learn the electrocardiographic waveforms of the plurality of leads in this manner, the noise detection model having high robustness against deviation of the attachment position of an electrode of the electrocardiograph can be generated.

### Learning of Arrhythmia Detection Model

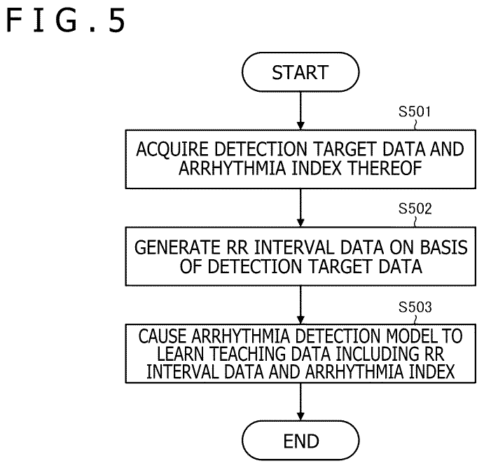

With reference to FIG. 5, a description is given of a learning method for causing a model for detecting whether arrhythmia has appeared in time-series data of a detection target to learn teaching data. In the following description, the time-series data of a detection target is represented as detection target data, and the model for detecting whether arrhythmia has appeared in the detection target data is represented as an arrhythmia detection model. The detection target data may be time-series data representing a biological signal, and may be time-series data representing, for example, an electrocardiographic waveform. The arrhythmia detection model outputs an index representing whether arrhythmia has appeared in the time-series data of the detection target. In the following description, the index representing whether arrhythmia has appeared is represented as an arrhythmia index. The arrhythmia index may be represented with two levels (for example, “arrhythmia is present” and “arrhythmia is absent”), or may be represented with three or more levels (for example, “high,” “middle,” and “low”). Instead of this, the arrhythmia index may be represented by the probability at which arrhythmia has appeared.

Each step in the method of FIG. 5 may be executed through execution of a program read out into the memory 102 by the processor 101. Instead of this, at least part of the steps in the method of FIG. 5 may be executed by a dedicated integrated circuit such as an ASIC or an FPGA. The method of FIG. 5 may be started in accordance with an instruction from a user of the computer 100.

In S501, the processor 101 acquires a training dataset used in machine learning of the arrhythmia detection model. The processor 101 may acquire the training dataset from an external server (for example, a database server) by using the communication device 105.

The training dataset for the arrhythmia detection model includes a plurality of pieces of training data. Each of the plurality of pieces of training data includes one piece of the detection target data (fifth time-series data) and the arrhythmia index representing whether arrhythmia has appeared in this detection target data. The detection target data is, for example, time-series data representing an electrocardiographic waveform measured by an electrocardiograph. The electrocardiograph that measures the electrocardiographic waveform may be either a Holter electrocardiograph or a polysomnography device, or may be an electrocardiograph of another type. The length of the detection target data is, for example, such a length as to include at least several times of pulsation, and may be, for example, approximately ten seconds. The detection target data may be a part extracted from an electrocardiographic waveform measured for a long period (for example, a sleeping period or one day or longer). The arrhythmia index of the detection target data may be an index decided by a person, or may be an index decided through execution of specific processing by a machine.

In S502, the processor 101 generates time-series data (sixth time-series data) in which a value of a respective one of a plurality of RR intervals of the detection target data continues for a period of time according to the RR interval immediately subsequent to the respective one of the RR intervals concerning each piece of the training data of the training dataset acquired in S501. In the following description, the time-series data in which a value of a respective one of a plurality of RR intervals of the detection target data continues for a period of time according to the RR interval immediately subsequent to the respective one of the RR intervals is represented as RR interval data.

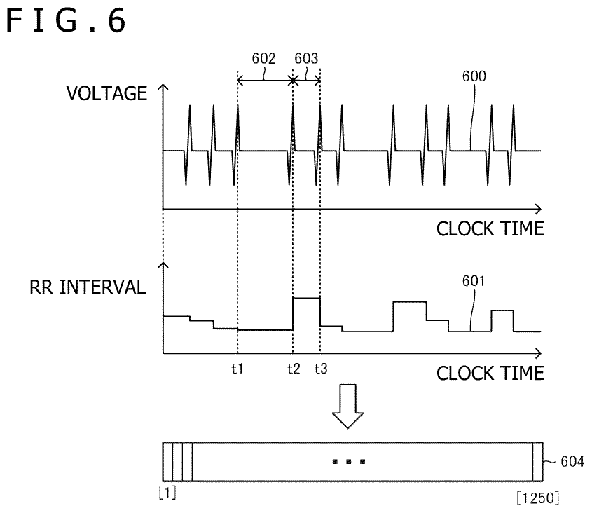

An example of detection target data 600 and RR interval data 601 is described with reference to FIG. 6. In FIG. 6, the detection target data 600 is represented by a graph in which the horizontal axis indicates the clock time and the vertical axis indicates a voltage, and the RR interval data 601 is represented by a graph in which the horizontal axis indicates the clock time and the vertical axis indicates the RR interval. In the example of FIG. 6, an R wave occurs in the detection target data 600 at a clock time t1. Then, the next R wave occurs in the detection target data 600 at a clock time t2, and the next R wave occurs in the detection target data 600 at a clock time t3. A time length 602 from the clock time t1 to the clock time t2 is one RR interval, and a time length 603 from the clock time t2 to the clock time t3 is the next RR interval.

The processor 101 sets the value at the clock time t2 concerning the RR interval data 601 to the time length 602, which is the latest RR interval at this time point. Thereafter, the processor 101 keeps the value of the RR interval data 601 at the time length 602 until the clock time t3. When the next R wave is detected at the clock time t3, the processor 101 changes the value of the RR interval data 601 to the time length 603, which is the latest RR interval at this time point. In this manner, the time during which the value of the RR interval data 601 is the time length 602 continues for the time length 603.

In S503, the processor 101 causes the arrhythmia detection model to learn teaching data (third teaching data) that includes the RR interval data generated in S502 as an input and includes the arrhythmia index as a correct answer concerning each piece of the training data of the training dataset acquired in S501. The data input to the arrhythmia detection model is referred to as input data. For example, the processor 101 calculates the difference between the arrhythmia index output from the arrhythmia detection model by inputting the input data to the arrhythmia detection model and the arrhythmia index of the correct answer concerning each piece of the training data of the training dataset, and updates a parameter of the arrhythmia detection model such that the sum of this difference across the plurality of pieces of the training data becomes small. Part of the plurality of pieces of the training data acquired in S501 may be used as validation data or test data of the arrhythmia detection model instead of being used as the teaching data.

An example of input data 604 to the arrhythmia detection model is described with reference to FIG. 6. The input data 604 is represented by a one-dimensional array. In the input data 604, the RR interval data 601 is represented by 1250 values. Instead of this, one piece of the RR interval data 601 may be represented by another number of values. For example, the 1250 values are decided by sampling one piece of the RR interval data 601 at a sampling cycle of 125 Hz.

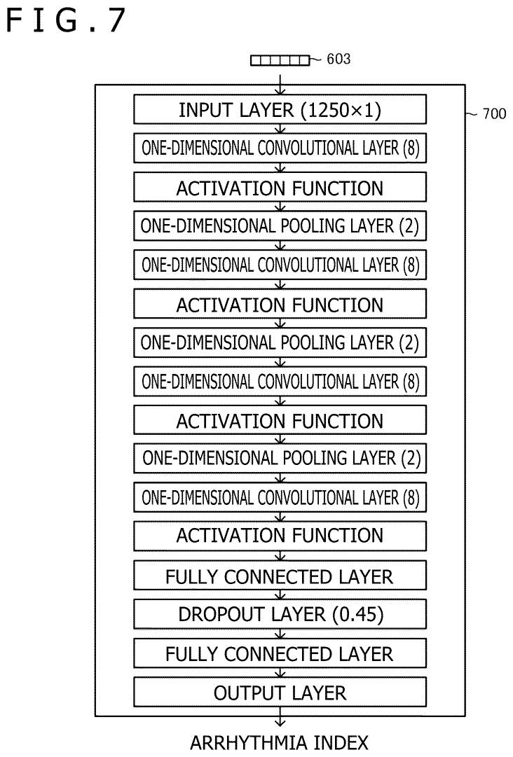

An example of a configuration of an arrhythmia detection model 700 is described with reference to FIG. 7. The arrhythmia detection model 700 includes a neural network (specifically, a CNN). Instead of this, the arrhythmia detection model 700 may include another model such as logistic regression or a support vector machine. Numerical values in parentheses given to an input layer of the arrhythmia detection model 700 represent the size of the input data 604. Numerical values in parentheses given to one-dimensional convolutional layers and one-dimensional pooling layers of the arrhythmia detection model 700 represent the window size. A numerical value in parentheses given to a dropout layer of the arrhythmia detection model 700 represents the dropout probability. A specific configuration of each layer of the arrhythmia detection model 700 may be an existing configuration, and thus detailed description thereof is omitted.

The noise detection model 400 is a two-dimensional CNN, and the arrhythmia detection model 700 is a one-dimensional CNN. Thus, typically, the processing load of the arrhythmia detection model 700 is lower than that of the noise detection model 400. The processing load may be evaluated on the basis of a processing time from input of the input data to the model to output of data from the model, or may be evaluated on the basis of the number of operations (for example, summation, multiplication, and the like) included in the model.

In machine learning of the above-described arrhythmia detection model 700, the above-described RR interval data 601 is used as the one piece of input data 604. In the RR interval data 601, adjacent two RR intervals are expressed in association with each other as the value of the RR interval data 601 and the duration of the value. Thus, by training the arrhythmia detection model 700 by using such input data 604, the arrhythmia detection model 700 with high accuracy of detection of arrhythmia can be generated.

The above-described method of FIG. 5 causes the model (arrhythmia detection model) for detecting whether arrhythmia has appeared in the detection target data to learn teaching data. Instead of this, the method of FIG. 5 can be used also for learning of teaching data by a model for detecting heart rate variability with use of the RR interval of an electrocardiographic waveform as an input, a model for detecting whether or not heart disease such as myocardial infarction or a pacemaker is present with use of the RR interval of an electrocardiographic waveform or another kind of information as an input, a model for detecting an abnormality including arrhythmia with use of a pulse wave as an input, a model for detecting a respiratory abnormality with use of a respiratory waveform as an input, or the like.

Similarly to the noise detection model, a single arrhythmia detection model may be trained by using electrocardiographic waveforms of a plurality of leads, or a different arrhythmia detection model may be trained concerning each lead.

### Analysis of Electrocardiographic Waveform

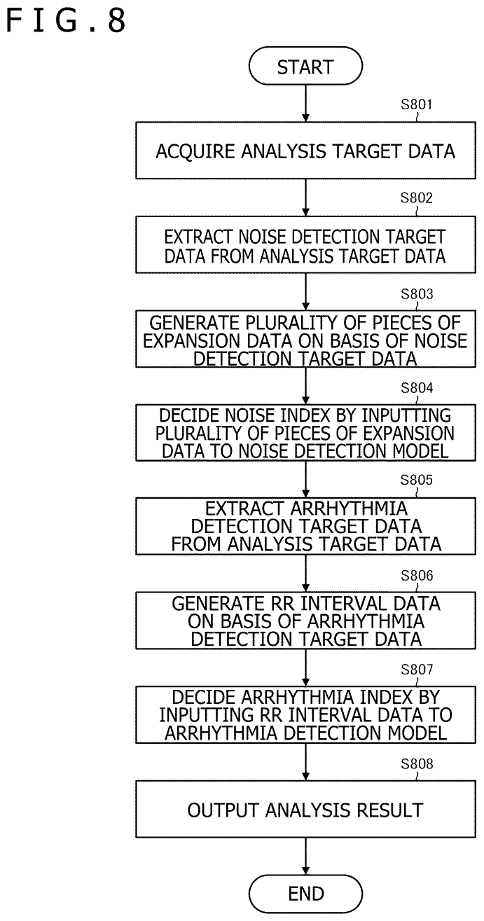

An analysis method for analyzing time-series data is described with reference to FIG. 8. In the following description, the time-series data of an analysis target is represented as analysis target data. The analysis target data may be time-series data representing a biological signal, and may be time-series data representing, for example, an electrocardiographic waveform.

Each step in the method of FIG. 8 may be executed through execution of a program read out into the memory 102 by the processor 101. Instead of this, at least part of the steps in the method of FIG. 8 may be executed by a dedicated integrated circuit such as an ASIC or an FPGA. The method of FIG. 8 may be started in accordance with an instruction from a user of the computer 100. Instead of this, the method of FIG. 8 may be periodically executed.

In S801, the processor 101 acquires the analysis target data. The processor 101 may acquire the analysis target data from an external server (for example, a database server) by using the communication device 105, or may acquire the analysis target data from an electrocardiograph. The analysis target data is, for example, time-series data representing an electrocardiographic waveform measured by an electrocardiograph. The electrocardiograph that measures the electrocardiographic waveform may be either a Holter electrocardiograph or a polysomnography device, or may be an electrocardiograph of another type. The analysis target data may be time-series data representing an electrocardiographic waveform measured for a long period (for example, a sleeping period or one day or longer).

In S802, the processor 101 extracts a part for detecting the noise amount from the analysis target data acquired in S801. Time-series data of the part extracted from the analysis target data in S802 is represented as noise detection target data. As described later, the noise amount of the noise detection target data is detected by using the noise detection model generated by the above-described method of FIG. 2. Thus, the length of the noise detection target data may be the same as the length of the detection target data for the noise detection model (for example, approximately two seconds). The processor 101 may extract a plurality of pieces of the noise detection target data from the analysis target data. In this case, the processor 101 executes the following S803 and S804 concerning each of the plurality of pieces of the noise detection target data.

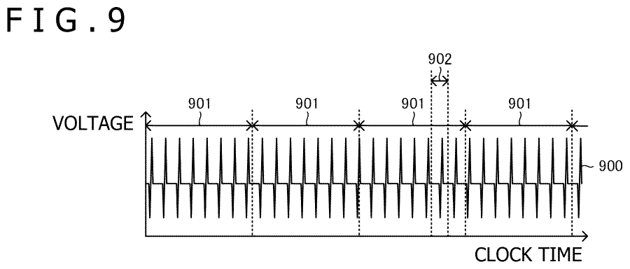

An example of the analysis target data and the noise detection target data is described with reference to FIG. 9. In FIG. 9, analysis target data 900 is represented by a graph in which the horizontal axis indicates the clock time and the vertical axis indicates a voltage. The processor 101 may divide the whole of the analysis target data 900 into the lengths of the detection target data for the noise detection model and extract each part resulting from the dividing as noise detection target data 902. Instead of this, the processor 101 may extract only a part that meets a specific condition in the analysis target data 900 as the noise detection target data 902. In the example of FIG. 9, the processor 101 extracts part of the analysis target data 900 as the noise detection target data 902. For example, concerning a part at which the amount of noise is obviously small and a part at which the amount of noise is obviously large in the analysis target data 900, whether or not noise exists can be detected without requiring to use the noise detection model. Thus, the processor 101 is not required to extract these parts as the noise detection target data 902. Further, the processor 101 may detect the noise amount of the analysis target data 900 by using a method involving a lower processing load than detection using the noise detection model and extract a part at which the noise amount is included in a predetermined range (for example, from a small amount to a middle amount, or from a middle amount to a large amount) as the noise detection target data 902.

In S803, the processor 101 generates a plurality of pieces of time-series data (eighth time-series data) different from each other on the basis of the noise detection target data similarly to S202 in FIG. 2. Specifically, the processor 101 generates a plurality of pieces of expansion data different from each other by executing denoising or noise addition for the noise detection target data. For example, similarly to S202 in FIG. 2, the plurality of pieces of expansion data may include both the expansion data generated by reducing a frequency component lower than a predetermined cutoff frequency (third cutoff frequency) from the detection target data and the expansion data generated by reducing a frequency component higher than a predetermined cutoff frequency (fourth cutoff frequency) from the detection target data, or may include only either one of them.

In S804, the processor 101 decides the noise index representing the noise amount included in the noise detection target data extracted in S802 by inputting data including the plurality of pieces of expansion data generated in S803 to the noise detection model generated by the method of FIG. 2. As described above, the noise detection model outputs the noise index when the data including the plurality of pieces of expansion data is input thereto.

In S805, the processor 101 extracts a part for detecting whether arrhythmia has appeared from the analysis target data acquired in S801. Time-series data of the part extracted from the analysis target data in S805 is represented as arrhythmia detection target data. As described later, whether arrhythmia has appeared in the arrhythmia detection target data is detected by using the arrhythmia detection model generated by the above-described method of FIG. 5. Thus, the length of the arrhythmia detection target data may be the same as the length of the detection target data for the arrhythmia detection model (for example, approximately ten seconds). The processor 101 may extract a plurality of pieces of the arrhythmia detection target data from the analysis target data. In this case, the processor 101 executes the following S806 and S807 concerning each of the plurality of pieces of the arrhythmia detection target data.

An example of the analysis target data and the arrhythmia detection target data is described with reference to FIG. 9 again. The processor 101 extracts part of the analysis target data 900 as arrhythmia detection target data 901 (eleventh time-series data). For example, the processor 101 divides the whole of the analysis target data 900 into the lengths of the detection target data for the arrhythmia detection model and extracts each part resulting from the dividing as the arrhythmia detection target data 901. Instead of this, the processor 101 may extract, as the arrhythmia detection target data 901, a part detected to have a noise amount included in a predetermined range (for example, a small amount or from a small amount to a middle amount) by the noise detection model in the analysis target data 900. Further, the processor 101 may specify a part at which arrhythmia is suspected from the part detected to have a noise amount included in the predetermined range by the noise detection model by using a method involving a lower processing load than detection using the arrhythmia detection model and extract this part as the arrhythmia detection target data 901. Moreover, the processor 101 may extract, as the arrhythmia detection target data 901, a part at which arrhythmia is suspected and specified by using a method involving a lower processing load than detection using the arrhythmia detection model, in the analysis target data 900.

In S806, similarly to S502 in FIG. 5, the processor 101 generates RR interval data (twelfth time-series data) in which a value of a respective one of a plurality of RR intervals of the arrhythmia detection target data extracted in S805 continues for a period of time according to the RR interval immediately subsequent to the respective one of the RR intervals.

In S807, the processor 101 decides the arrhythmia index representing whether arrhythmia has appeared in the arrhythmia detection target data extracted in S805 by inputting the RR interval data generated in S806 to the arrhythmia detection model generated by the method of FIG. 5. As described above, the arrhythmia detection model outputs the arrhythmia index when the RR interval data is input thereto.

In S808, the processor 101 outputs an analysis result including only either one or both of the noise index decided in S804 and the arrhythmia index decided in S807. For example, the processor 101 may store the analysis result in the storage device 106, display the analysis result on the output device 104 (for example, a display), or transmit the analysis result to an external device through the communication device 105. The output of the analysis result may include associating the noise detection target data with the noise index thereof and outputting them, and associating the arrhythmia detection target data with the arrhythmia index thereof and outputting them. For example, in the graph representing the analysis target data 900 in FIG. 9, only either one or both of a period in which noise is large and a period in which the possibility that arrhythmia has appeared is high may be indicated.

When the analysis target data acquired in the above-described S801 represents electrocardiographic waveforms of a plurality of leads, the processor 101 may execute S802 to S807 concerning at least part of the electrocardiographic waveform of each lead and execute S808 on the basis of the execution result. For example, the analysis target data may represent an electrocardiographic waveform of the CM5 lead (third lead) and an electrocardiographic waveform of the NASA lead (fourth lead).

Specifically, in the above-described S803, the processor 101 generates a plurality of pieces of time-series data (eighth time-series data) different from each other on the basis of the noise detection target data (seventh time-series data) of the CM5 lead, and generates a plurality of pieces of time-series data (tenth time-series data) different from each other on the basis of the noise detection target data (ninth time-series data) of the NASA lead. In the above-described S804, the processor 101 decides the noise index (third noise index) of the CM5 lead by inputting data including the plurality of pieces of time-series data of the CM5 lead to the noise detection model, and decides the noise index (fourth noise index) of the CM5 lead by inputting data including the plurality of pieces of time-series data of the NASA lead to the noise detection model.

Further, the processor 101 extracts the arrhythmia detection target data from the analysis target data in the above-described S805, and generates the RR interval data of the CM5 lead and the RR interval data of the NASA lead in the above-described S806. In the above-described S807, the processor 101 decides the arrhythmia index of the CM5 lead by inputting the RR interval data of the CM5 lead to the arrhythmia detection model, and decides the arrhythmia index of the NASA lead by inputting the RR interval data of the NASA lead to the arrhythmia detection model.

In the method of FIG. 8, the arrhythmia detection is executed in S805 to S807 after the noise detection is executed in S802 to S804. Instead of this, the arrhythmia detection may be executed before the noise detection, or the noise detection and the arrhythmia detection may be concurrently executed. In a case in which the arrhythmia detection is executed after the noise detection is executed, the arrhythmia detection may be executed only concerning a period detected to involve a small noise amount in the noise detection in the analysis target data.

In the above description, the learning method of FIG. 2, the learning method of FIG. 5, and the analysis method of FIG. 8 are executed by the same computer 100. Instead of this, these methods may be executed by different computers.

### Summary of Embodiment

### (Item 1)

A learning device including:
a processor; and
a memory, in which
the memory stores a program for causing the processor to execute
   acquiring first time-series data representing a biological waveform and a noise index representing a noise amount included in the first time-series data,
   generating a plurality of pieces of second time-series data different from each other by executing denoising or noise addition for the first time-series data, and
   causing a noise detection model to learn teaching data that includes data including the plurality of pieces of second time-series data as an input and includes the noise index as a correct answer.

### (Item 2)

The learning device according to Item 1, in which the plurality of pieces of second time-series data include time-series data generated by executing denoising for the first time-series data.

### (Item 3)

The learning device according to Item 1 or 2, in which the plurality of pieces of second time-series data include time-series data generated by executing noise addition for the first time-series data.

### (Item 4)

The learning device according to any one of Items 1 to 3, in which
the plurality of pieces of second time-series data include
   time-series data generated by reducing a frequency component lower than a first cutoff frequency from the first time-series data, and
   time-series data generated by reducing a frequency component higher than a second cutoff frequency from the first time-series data.

### (Item 5)

The learning device according to Item 4, in which the plurality of pieces of second time-series data further include time-series data generated by executing noise addition for the first time-series data.

### (Item 6)

The learning device according to any one of Items 1 to 5, in which
the first time-series data represents an electrocardiographic waveform of a first lead, and
the program causes the processor to further execute
   acquiring third time-series data representing an electrocardiographic waveform of a second lead different from the first lead and a second noise index representing a noise amount included in the third time-series data,
   generating a plurality of pieces of fourth time-series data different from each other by executing denoising or noise addition for the third time-series data, and
   causing the noise detection model to learn second teaching data that includes data including the plurality of pieces of fourth time-series data as an input and includes the second noise index as a correct answer.

### (Item 7)

The learning device according to any one of Items 1 to 6, in which
the program causes the processor to further execute
   acquiring fifth time-series data representing an electrocardiographic waveform and an arrhythmia index representing whether arrhythmia has appeared in the fifth time-series data,
   generating sixth time-series data in which a value of a respective one of a plurality of RR intervals in the fifth time-series data continues for a period of time according to the RR interval immediately subsequent to the respective one of the RR intervals, and
   causing an arrhythmia detection model to learn third teaching data that includes the sixth time-series data as an input and includes the arrhythmia index as a correct answer.

### (Item 8)

The learning device according to Item 7, in which a processing load of the arrhythmia detection model is lower than a processing load of the noise detection model.

### (Item 9)

A learning method executed by a computer, including:
acquiring, by an acquisition section of the computer, first time-series data representing a biological waveform and a noise index representing a noise amount included in the first time-series data;
generating, by a generation section of the computer, a plurality of pieces of second time-series data different from each other by executing denoising or noise addition for the first time-series data; and
causing, by a learning section of the computer, a noise detection model to learn teaching data that includes data including the plurality of pieces of second time-series data as an input and includes the noise index as a correct answer.

### (Item 10)

A non-transitory storage medium storing a program for causing a computer to execute the acquiring, the generating, and the causing in the learning method according to Item 9.

### (Item 11)

An analysis device including:
a processor; and
a memory, in which
the memory stores a program for causing the processor to execute
   acquiring seventh time-series data representing a biological waveform,
   generating a plurality of pieces of eighth time-series data different from each other by executing denoising or noise addition for the seventh time-series data, and
   deciding a third noise index representing a noise amount included in the seventh time-series data, by inputting data including the plurality of pieces of eighth time-series data to a noise detection model.

### (Item 12)

The analysis device according to Item 11, in which the plurality of pieces of eighth time-series data include time-series data generated by executing denoising for the seventh time-series data.

### (Item 13)

The analysis device according to Item 11 or 12, in which the plurality of pieces of eighth time-series data include time-series data generated by executing noise addition for the seventh time-series data.

### (Item 14)

The analysis device according to any one of Items 11 to 13, in which
the plurality of pieces of eighth time-series data include
   time-series data generated by reducing a frequency component lower than a third cutoff frequency from the seventh time-series data, and
   time-series data generated by reducing a frequency component higher than a fourth cutoff frequency from the seventh time-series data.

### (Item 15)

The analysis device according to Item 14, in which the plurality of pieces of eighth time-series data further include time-series data generated by executing noise addition for the seventh time-series data.

### (Item 16)

The analysis device according to any one of Items 11 to 15, in which
the seventh time-series data represents an electrocardiographic waveform of a third lead, and
the program causes the processor to further execute
   acquiring ninth time-series data representing an electrocardiographic waveform of a fourth lead different from the third lead,
   generating a plurality of pieces of tenth time-series data different from each other by executing denoising or noise addition for the ninth time-series data, and
   deciding a fourth noise index representing a noise amount included in the ninth time-series data, by inputting data including the plurality of pieces of tenth time-series data to the noise detection model.

### (Item 17)

The analysis device according to any one of Items 11 to 16, in which
the program causes the processor to further execute
   acquiring eleventh time-series data representing an electrocardiographic waveform,
   generating twelfth time-series data in which a value of a respective one of a plurality of RR intervals in the eleventh time-series data continues for a period of time according to the RR interval immediately subsequent to the respective one of the RR intervals, and
   deciding an arrhythmia index representing whether arrhythmia has appeared in the eleventh time-series data, by inputting the twelfth time-series data to an arrhythmia detection model.

### (Item 18)

The analysis device according to Item 17, in which a processing load of the arrhythmia detection model is lower than a processing load of the noise detection model.

### (Item 19)

An analysis method executed by a computer, including:
acquiring, by an acquisition section of the computer, seventh time-series data representing a biological waveform;
generating, by a generation section of the computer, a plurality of pieces of eighth time-series data different from each other by executing denoising or noise addition for the seventh time-series data; and
deciding, by a decision section of the computer, a third noise index representing a noise amount included in the seventh time-series data, by inputting data including the plurality of pieces of eighth time-series data to a noise detection model.

### (Item 20)

A non-transitory storage medium storing a program for causing a computer to execute the acquiring, the generating, and the deciding in the analysis method according to Item 19.

The disclosure is not limited to the above-described embodiment, and various modifications and changes are possible within the scope of the gist of the disclosure.

What is claimed is:

1. A learning device comprising:
a processor; and
a memory, wherein
the memory stores a program for causing the processor to execute
acquiring first time-series data representing a biological waveform and a noise index representing a noise amount included in the first time-series data,
generating a plurality of pieces of second time-series data different from each other by executing denoising or noise addition for the first time-series data, and
causing a noise detection model to learn teaching data that includes data including the plurality of pieces of second time-series data as an input and includes the noise index as a correct answer.

2. The learning device according to claim 1, wherein the plurality of pieces of second time-series data include time-series data generated by executing denoising for the first time-series data.

3. The learning device according to claim 1, wherein the plurality of pieces of second time-series data include time-series data generated by executing noise addition for the first time-series data.

4. The learning device according to claim 1, wherein
the plurality of pieces of second time-series data include
time-series data generated by reducing a frequency component lower than a first cutoff frequency from the first time-series data, and
time-series data generated by reducing a frequency component higher than a second cutoff frequency from the first time-series data.

5. The learning device according to claim 4, wherein the plurality of pieces of second time-series data further include time-series data generated by executing noise addition for the first time-series data.

6. The learning device according to claim 1, wherein
the first time-series data represents an electrocardiographic waveform of a first lead, and
the program causes the processor to further execute
acquiring third time-series data representing an electrocardiographic waveform of a second lead different from the first lead and a second noise index representing a noise amount included in the third time-series data,
generating a plurality of pieces of fourth time-series data different from each other by executing denoising or noise addition for the third time-series data, and
causing the noise detection model to learn second teaching data that includes data including the plurality of pieces of fourth time-series data as an input and includes the second noise index as a correct answer.

7. The learning device according to claim 1, wherein
the program causes the processor to further execute
acquiring fifth time-series data representing an electrocardiographic waveform and an arrhythmia index representing whether arrhythmia has appeared in the fifth time-series data,
generating sixth time-series data in which a value of a respective one of a plurality of RR intervals in the fifth time-series data continues for a period of time according to the RR interval immediately subsequent to the respective one of the RR intervals, and
causing an arrhythmia detection model to learn third teaching data that includes the sixth time-series data as an input and includes the arrhythmia index as a correct answer.

8. The learning device according to claim 7, wherein a processing load of the arrhythmia detection model is lower than a processing load of the noise detection model.

9. An analysis device comprising:
a processor; and
a memory, wherein
the memory stores a program for causing the processor to execute
acquiring seventh time-series data representing a biological waveform,
generating a plurality of pieces of eighth time-series data different from each other by executing denoising or noise addition for the seventh time-series data, and
deciding a third noise index representing a noise amount included in the seventh time-series data, by inputting data including the plurality of pieces of eighth time-series data to a noise detection model.

10. The analysis device according to claim 9, wherein the plurality of pieces of eighth time-series data include time-series data generated by executing denoising for the seventh time-series data.

11. The analysis device according to claim 9, wherein the plurality of pieces of eighth time-series data include time-series data generated by executing noise addition for the seventh time-series data.

12. The analysis device according to claim 9, wherein
the plurality of pieces of eighth time-series data include
time-series data generated by reducing a frequency component lower than a third cutoff frequency from the seventh time-series data, and
time-series data generated by reducing a frequency component higher than a fourth cutoff frequency from the seventh time-series data.

13. The analysis device according to claim 12, wherein the plurality of pieces of eighth time-series data further include time-series data generated by executing noise addition for the seventh time-series data.

14. The analysis device according to claim 9, wherein
the seventh time-series data represents an electrocardiographic waveform of a third lead, and
the program causes the processor to further execute
acquiring ninth time-series data representing an electrocardiographic waveform of a fourth lead different from the third lead,
generating a plurality of pieces of tenth time-series data different from each other by executing denoising or noise addition for the ninth time-series data, and
deciding a fourth noise index representing a noise amount included in the ninth time-series data, by inputting data including the plurality of pieces of tenth time-series data to the noise detection model.

15. The analysis device according to claim 9, wherein
the program causes the processor to further execute
acquiring eleventh time-series data representing an electrocardiographic waveform,
generating twelfth time-series data in which a value of a respective one of a plurality of RR intervals in the eleventh time-series data continues for a period of time according to the RR interval immediately subsequent to the respective one of the RR intervals, and
deciding an arrhythmia index representing whether arrhythmia has appeared in the eleventh time-series data, by inputting the twelfth time-series data to an arrhythmia detection model.

16. The analysis device according to claim 15, wherein a processing load of the arrhythmia detection model is lower than a processing load of the noise detection model.

17. An analysis method executed by a computer, comprising:
acquiring, by an acquisition section of the computer, seventh time-series data representing a biological waveform;
generating, by a generation section of the computer, a plurality of pieces of eighth time-series data different from each other by executing denoising or noise addition for the seventh time-series data; and
deciding, by a decision section of the computer, a third noise index representing a noise amount included in the seventh time-series data, by inputting data including the plurality of pieces of eighth time-series data to a noise detection model.

18. A non-transitory storage medium storing a program for causing a computer to execute the acquiring, the generating, and the deciding in the analysis method according to claim 17.
